(19) United States
(12) Patent Application Publication
(10) Pub. No.: US 2010/0099126 A1
(43) Pub. Date: Apr. 22, 2010

(54) METHOD OF CALCULATING ENZYMATIC REACTION RATE, COMPUTER PROGRAM PRODUCT AND METHOD OF DETERMINING AMOUNT OF ENZYME IN SAMPLE

(75) Inventors: Do Gyoon KIM, Yongin-si (KR); Hyun Min KIM, Gwangju-si (KR)

Correspondence Address: SUGHRUE MION, PLLC, 2100 PENNSYLVANIA AVENUE, N.W., SUITE 800, WASHINGTON, DC, 20037, US

(73) Assignee: Samsung Electronics Co., Ltd., Suwon-si (KR)

(21) Appl. No.: 12/576,577

(22) Filed: Oct. 9, 2009

(30) Foreign Application Priority Data
Oct. 20, 2008 (KR) 10-2008-0102560

(51) Int. Cl. C12Q 1/48; C12Q 1/34

(52) U.S. Cl. 435/15; 435/18

(57) ABSTRACT

A method of calculating an enzymatic reaction rate using the largest frequency value of a slope of a sub-group, a computer program product capable of performing the method of calculating an enzymatic reaction rate, and a method of determining the amount of an enzyme in a sample are provided.

### CROSS-REFERENCE TO RELATED APPLICATIONS

This application claims priority from Korean Patent Application No. 10-2008-0102560, filed on Oct. 20, 2008, in the Korean Intellectual Property Office, the disclosure of which is incorporated herein in its entirety by reference.

### BACKGROUND

1. Field

Methods consistent with the present inventive concept relate to calculating an enzymatic reaction rate and determining the amount of an enzyme in a sample.

2. Description of the Related Art

Various methods of measuring the amount of an enzyme in a sample are known. A colorimetric method is an example of these methods. In the colorimetric method, an enzyme is reacted with a substrate to obtain a reaction product and an optical signal such as a fluorescence signal is obtained directly or indirectly from the reaction product, and the amount of the enzyme in the sample can be measured by using a relationship between the amplitude of the optical signal and the amount of the enzyme. Also, in the colorimetric method, an enzymatic reaction rate can be calculated.

As a first step of the colorimetric method, a graph of absorbance, with respect to time, of a reaction product of the enzyme and the substrate is obtained. With respect to a linear portion of the graph, an absorbance change rate is equal to an absorbance change value per time. Then, the absorbance change value is multiplied by a predetermined factor which can be obtained using a reference sample in order to measure the amount of the enzyme in the sample. The method of measuring the amount of an enzyme in a sample using absorbance may have the following problems. First, if there is a variation (that is, noise) when absorbance is measured with respect to time, the obtained enzymatic reaction rate can be higher or lower than the actual enzymatic reaction rate. In addition, if the concentration of the enzyme in the sample is high, the substrate can be completely consumed before the measurement time has lapsed and thus, the measured enzymatic reaction rate may be erroneous. Furthermore, if the change of the concentration of the substrate with respect to time, for example, the change in absorbance, is not linear during a absorbance measurement period, the measured enzymatic reaction rate may be erroneous.

Accordingly, there is still a need to develop a method of uniformly, precisely calculating an enzymatic reaction rate.

### SUMMARY

Disclosed herein are one or more exemplary embodiments providing a method of efficiently, precisely calculating an enzymatic reaction rate. According to an exemplary embodiment, there is provided a method of calculating a reaction rate of a first substance contained in a sample, the method including: obtaining an optical signal, with respect to a reaction time, of a mixture including the first substance and a second substance, wherein a reaction between the first substance and the second substance generates the optical signal; dividing the optical signal into sub-groups of the optical signal based on a reaction time interval; calculating a slope with respect to each sub-group of the optical signal and the reaction time; obtaining a frequency of the calculated slope of each sub-group with respect to a slope value interval; subtracting a slope value (Sn) of each sub-group from a slope value (Sf) that corresponds to a largest frequency value among the obtained frequency values, thereby obtaining differences between Sf and Sn; adding up the differences with respect to a predetermined number of continuous sub-groups and selecting a continuous sub-group section that corresponds to a minimum differences sum among the added-up differences sums; and calculating a slope of the selected continuous sub-group section and determining the calculated slope as the reaction rate.

In an exemplary embodiment, the first substance is an enzyme and the second substance is a substrate of the enzyme. The second substance may be added to the sample containing the first substance.

Also disclosed herein are one or more exemplary embodiments providing a computer program product for efficiently, precisely calculating an enzymatic reaction rate.

In addition, disclosed herein are one or more exemplary embodiments providing a method of efficiently, precisely determining the amount of an enzyme in a sample. According to an exemplary embodiment, there is provided A method of determining an amount of an enzyme in a sample, the method including: calculating an enzymatic reaction rate with respect to a sample comprising an enzyme by using the method of the above-described method; and comparing the calculated enzymatic reaction rate with a reference enzymatic reaction rate with respect to an enzyme concentration which is obtained using a control sample containing a known concentration of the enzyme, in order to determine the amount of the enzyme.

### BRIEF DESCRIPTION OF THE DRAWINGS

These and/or other aspects, advantages and features of this disclosure will become apparent and more readily appreciated from the following description of the exemplary embodiments, taken in conjunction with the accompanying drawings of which:

FIG. 1 is a graph of amplitude of an optical signal (i.e., absorbance), with respect to a reaction time, of a mixture including an enzyme and a substrate;

FIG. 2 is a diagram illustrating a case in which optical signals of FIG. 1 are divided into sub-groups of optical signals based on a predetermined reaction time interval;

FIG. 3 is a graph of a frequency of slopes with respect to the sub groups shown in FIG. 2;

FIG. 4 is a graph illustrating an example of a section that is selected for obtaining a slope; and

FIGS. 5A through 5I are graphs illustrating results obtained by assaying a sample containing AST 9 times using a method according to an exemplary embodiment, wherein the dotted line illustrates measured absorbance, the bold solid line represents a slope obtained using a method according to an exemplary example, and a thin solid line represents a slope obtained using a conventional method.

### DETAILED DESCRIPTION

Reference will now be made in detail to exemplary embodiments with reference to the accompanying drawings. In this regard, the present exemplary embodiments may have different forms and should not be construed as being limited to the descriptions set forth herein. Accordingly, the exemplary embodiments are merely described below, by referring to the figures, to explain aspects of the present description.

An exemplary embodiment provides a method of calculating a reaction rate of a first substance, wherein the method includes obtaining an optical signal, with respect to a reaction time, of a mixture including the first substance and a second substance; dividing the obtained optical signal into sub-groups of the optical signal based on a reaction time interval; calculating a slope with respect to each sub-group of the optical signal and the reaction time; obtaining a frequency of the calculated slope of each sub-group with respect to a slope value interval; subtracting a slope value (Sn) of each sub-group from a slope value (Sf) that corresponds to the largest frequency value among the obtained frequency values, thereby obtaining the difference between Sf and Sn; adding up such differences with respect to a predetermined number of continuous sub-groups and selecting a continuous sub-group section that corresponds to the minimum differences sum among obtained differences sums; and calculating a slope of the selected continuous sub-group section and determining the calculated slope as the reaction rate of the first substance. In an exemplary embodiment, the first substrate may be an enzyme and the second substance may a substrate of the enzyme. The second substance can be added to the sample, which contains the first substance, to generate the optical signal upon an action of the first substance on the second substance.

The method of calculating an enzymatic reaction rate may include obtaining an optical signal, with respect to a reaction time, of a mixture including an enzyme and a substrate.

The enzyme may be alkaline phosphatase or aminotransferase. The enzyme may be included in a sample, for example, a biological sample. For example, the sample may be selected from the group consisting of blood, tissues, salvia, urine, and a body fluid. The substrate may be appropriately selected according to an enzyme used. The substrate may generate an optical signal or may be indirectly detectable by an optical signal.

The optical signal may be, for example, a fluorescent signal. The optical signal may be represented by an optical density. The optical signal may be obtained by absorbance measurement, permeability measurement, or radiation measurement.

The optical signal is measured with respect to time. The optical signal may be continuously measured with respect to time. Alternatively, the optical signal may be measured at a plurality of particular time points, and the obtained optical signal may be extrapolated by curve-fitting. The optical signal measured with respect to time may form an optical signal profile with respect to time. In addition, the optical signal measured with respect to time may be a digital signal to be processed by a computer.

The method of calculating an enzymatic reaction rate may also include dividing the obtained optical signal into sub-groups of the optical signal based on a reaction time interval.

The reaction time interval may be appropriately selected. The reaction time interval may be a reaction time interval by which the entire reaction time is divided into 5 to 30 sections. The division may be a division of the optical signal profile with respect to time into a plurality of sections.

The method of calculating an enzymatic reaction rate may also include calculating a slope with respect to each sub-group of the optical signal and the reaction time. The slope may be calculated using a method such as a regression analysis method or a robust estimation method. The slope may be calculated using, for example, the following equations.

$$\{ \begin{matrix}a = \frac{( \sum y ) ( \sum {x}^{2} ) - ( \sum x ) ( \sum xy )}{n \sum {x}^{2} - {( \sum x )}^{2}} \\ b = \frac{n \sum xy - ( \sum x ) ( \sum y )}{n \sum {x}^{2} - {( \sum x )}^{2}}\end{matrix}$$

The equations illustrated above are linear regression analysis calculation equations with respect to a data set (x₁,y₁), (x₂,y₂), . . . , (xₙ,yₙ), Σ| denotes a sum, and a and b denote a y-intercept and a slope, respectively.

The method of calculating an enzymatic reaction rate may also include obtaining a frequency of the calculated slope of each sub-group with respect to a slope value interval. The slope value interval may be appropriately determined based on a slope value distribution. The frequency may be represented by the number of sub-groups included in the slope value interval, for example, 0.5, 1.0, or 1.5 sections.

The method of calculating an enzymatic reaction rate may also include subtracting a slope value (S1, S2, S3, . . . , Sn) of each sub-group from a slope value (Sf) that corresponds to the largest frequency value among the obtained frequency values, thereby obtaining the difference between Sf and Sn.

The slope value may be any value in a slope section as long as the slope value is positioned at the same site in each slope section (that is, a corresponding position.) For example, the slope value may be a middle value in each slope section.

The method of calculating an enzymatic reaction rate may also include adding up such differences with respect to a predetermined number of continuous sub-groups and selecting a continuous sub-group section that corresponds to the minimum differences sum among the obtained differences sums.

The predetermined number of continuous sub-groups may vary according to the size of a reaction time period for which an enzymatic reaction rate is to be calculated. For example, when the obtained optical signal is divided into 10 sub-groups, an enzymatic reaction rate for a reaction time period corresponding to half the entire reaction time can be measured by selecting five of the 10 sub-groups, and an enzymatic reaction rate for a reaction time period corresponding to two-fifth of the entire reaction time can be measured by selecting four of the 10 sub-groups.

The method of calculating an enzymatic reaction rate may also include calculating a slope of the selected reaction time period and determining the calculated slope as an enzymatic reaction rate.

The method of calculating an enzymatic reaction rate may further include displaying the determined enzymatic reaction rate to a user. The displaying may be performed by displaying the determined enzymatic reaction rate on a display device such as a liquid crystal display (LCD), a light emitting diode (LED) display, an organic light emitting diode (OLED) display, and a cathode ray tube (CRT) monitor. The determined enzymatic reaction rate may be displayed in a digital numeric form, a graph, or a sign.

The method of calculating an enzymatic reaction rate may be realized with hardware, software, or combinations thereof. The method of calculating an enzymatic reaction rate may be realized with at least one computer system. Accordingly, an exemplary embodiment provides a computer system that performs a function corresponding to the method as described above.

Another exemplary embodiment provides a computer-readable recording medium having recorded thereon a program for executing the method as described above.

The computer-readable recording medium may be a storage medium such as a random access memory (RAM), a read-only memory (ROM), a compact disk (CD), a floppy disk, or a flash memory.

Another exemplary embodiment provides a computer program product including a computer-readable recording medium having recorded thereon a program for executing the method as described above.

The computer program product may enable an application program to operate in a computer that is used to calculate an enzymatic reaction rate according to the method as described above.

The computer program product may include a processor. The processor may be connected to a communications infrastructure such as a communications bus or a network.

The computer program product may include a graphic, a text, and a display interface that transfers data transmitted from the communications infrastructure, in order to display the data on a display unit.

The computer program product may include a main memory and a second memory. For example, the main memory may be a RAM. The second memory may be, for example, a hard disk drive and/or a removable storage drive such as a floppy disk drive, a magnetic tape drive, or an optical disk drive. The removable storage drive reads and writes on a removable storage unit by using well-known methods. Examples of the removable storage unit include a floppy disk, a magnetic tape, and an optical disk which are read and written by the removable storage drive. The removable storage unit may include computer software and/or a computer-readable storage medium that stores data.

In the present specification, the “computer-readable recording medium” include a signal and a medium such as a removable storage medium or a hard disk. The computer program products are used to provide software to a computer system.

A program or computer program (also called computer control logic) may be stored in a main memory and/or a secondary memory. The program or computer program may be received through a communications interface. If the program or computer program operates, all or a part of the method as described above may be performed by a computer system according to an exemplary embodiment.

Another exemplary embodiment provides a method of determining an amount of an enzyme in a sample, wherein the method includes: calculating an enzymatic reaction rate with respect to a sample including the enzyme by using the method as described above; and comparing the calculated enzymatic reaction rate with a reference enzymatic reaction rate with respect to an enzyme concentration which is obtained using a control sample containing a known concentration of the enzyme, in order to determine the amount of the enzyme.

The method of determining the amount of an enzyme in a sample may include calculating an enzymatic reaction rate with respect to the sample including the enzyme by using the method as described above.

The sample may be a biological sample. For example, the sample may be selected from the group consisting of blood, tissues, salvia, urine, and a body fluid.

The enzyme may be alkaline phosphatase or aminotransferase.

The method of determining the amount of an enzyme in a sample may also include comparing the calculated enzymatic reaction rate with a reference enzymatic reaction rate with respect to an enzyme concentration which is obtained using a control sample containing a known concentration of the enzyme, in order to determine the amount of the enzyme.

The reference enzymatic reaction rate with respect to the enzyme concentration may be calculated using the control sample containing a known concentration of the enzyme and the method as described above. The control sample may have the same composition as or a similar composition to the sample to be tested for the enzyme reaction rate, except that the control sample contains the enzyme in a known concentration. The similar composition means that at least one feature of pH, a salt concentration, and an ion concentration is similar to corresponding features in the control sample.

The amount of the enzyme in the sample can be determined by finding a reference enzyme concentration corresponding to an enzymatic reaction rate calculated with respect to a test sample by referring to a relationship between the enzyme concentration and the enzymatic reaction rate.

One or more exemplary embodiments will be described in further detail with reference to the following examples. The following examples are for illustrative purposes only and are not intended to limit the scope of the present inventive concept.

FIG. 1 is a graph of an optical signal, with respect to a reaction time, of a mixture including an enzyme and a substrate. Referring to FIG. 1, the optical signal can be obtained using, for example, the following method. In the presence of alanine transaminase (ALT), which is also called serum glutamate pyruvate transaminase (SGPT), NAD⁺, alanine and α-ketoglutarate are reacted and converted into NADH, pyruvate and glutamate. Then, in the presence of lactate dehydrogenase, the NADH and the pyruvate are reacted and converted into NAD⁺ and lactate. Then, absorbance of the NAD⁺ is measured at a wavelength of 340 nm. The absorbance of the NAD⁺ measured at a wavelength of 340 nm is similar to that of ALT in the sample. The graph of FIG. 1 may be drawn by curve-fitting optical signals measured at discontinuous points.

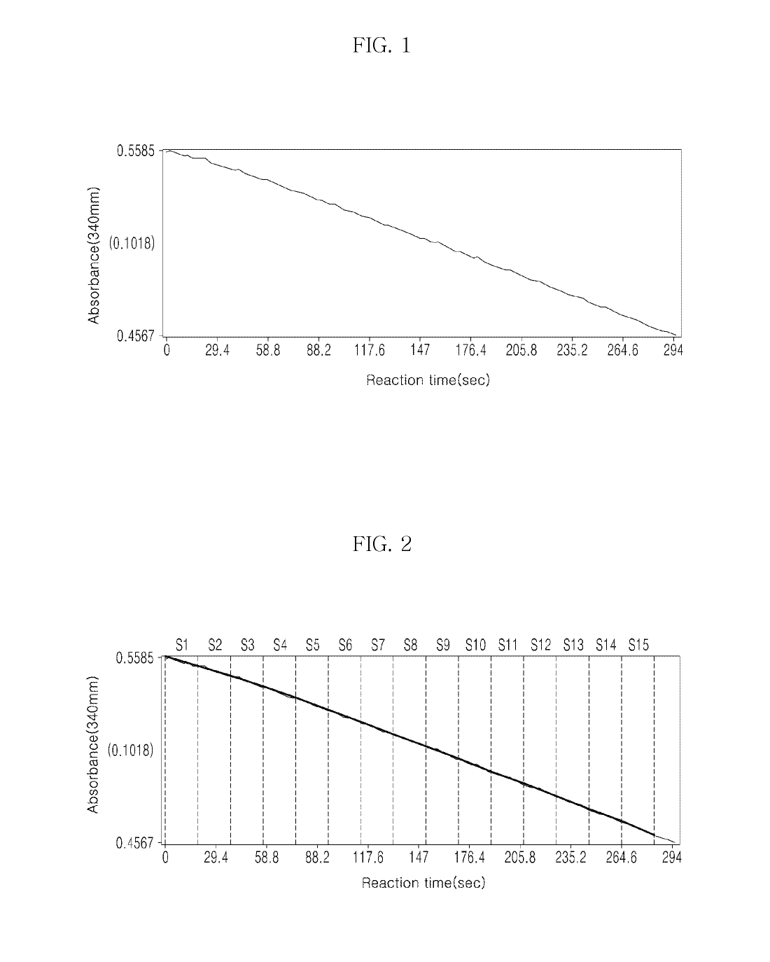

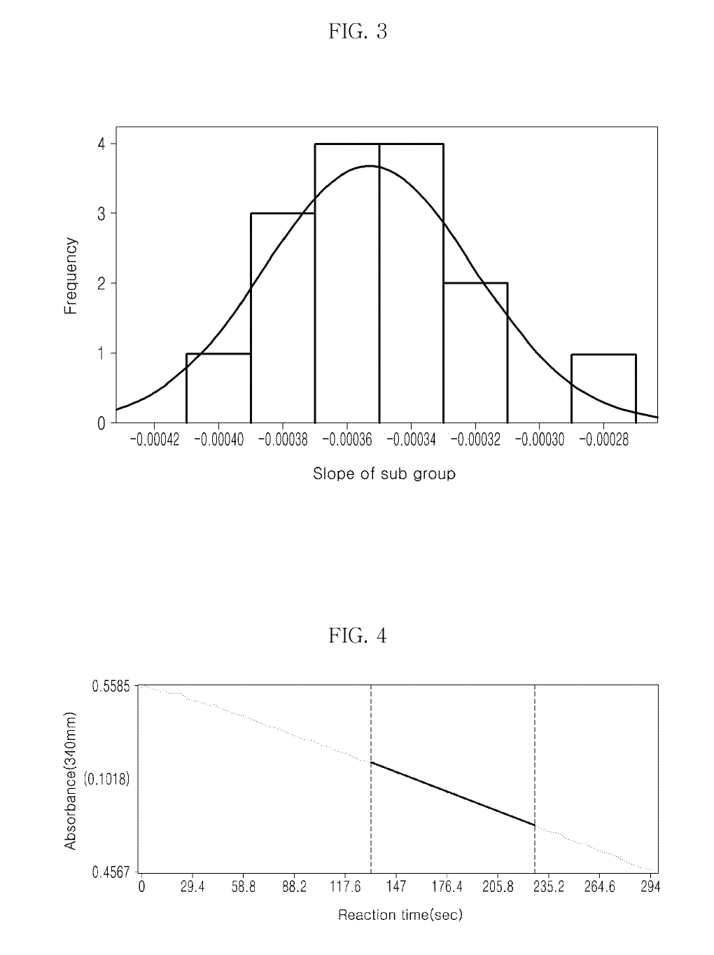

FIG. 2 is a diagram illustrating a case in which the optical signal of FIG. 1 is divided into sub-groups of the optical signal based on a predetermined reaction time interval. Referring to FIG. 2, the absorbance profile illustrated in FIG. 1 is divided into 15 sub-groups based on a reaction time interval of 18.9 seconds. Then, a slope of each sub-group is calculated. The slope may be calculated using a method such as a regression analysis method or a robust estimation method. The slope may be calculated using, for example, the following equations.

$$\{ \begin{matrix}a = \frac{( \sum y ) ( \sum {x}^{2} ) - ( \sum x ) ( \sum xy )}{n \sum {x}^{2} - {( \sum x )}^{2}} \\ b = \frac{n \sum xy - ( \sum x ) ( \sum y )}{n \sum {x}^{2} - {( \sum x )}^{2}}\end{matrix}$$

The equations illustrated above are linear regression analysis calculation equations with respect to a data set (x₁,y₁), (x₂,y₂), . . . , (xₙ,yₙ), Σ denotes a sum, and a and b denote a y-intercept and a slope, respectively.

In FIG. 2, the slope is a linear slope with respect to respective sub-groups obtained by a regression analysis. In FIG. 2, a bold solid line represents a slope calculated with respect to the entire section from section S1 to S15.

FIG. 3 is a graph of a frequency of slopes with respect to the sub-groups shown in FIG. 2 based on a predetermined slope interval of the sub groups. Referring to FIG. 3, Sf denotes a slope value corresponding to the largest frequency value. The slope value is not limited as long as a slope value at a uniform position in respective slope sections, that is, a value at the corresponding position in respective slope sections is selected. For example, the slope value may be a middle value. Although in FIG. 3 the slope interval value is 0.00002, the slope interval value may be appropriately selected.

As illustrated in FIG. 3, the Sf is calculated, the Sf is subtracted by Sn that is a slope value of each sub-group, thereby obtaining a difference value Dn. Herein, if at least two sub-groups have the largest frequency value, one of the sub-groups can be selected.

$$D 1 = Sf - S 1$$

$$D 2 = Sf - S 2$$

$$D 3 = Sf - S 3$$

$$…$$

$$Dn = Sf - Sn ,$$

wherein n denotes the number of sub-groups.

Then, difference values Dn with respect to predetermined numbers of continuous sub-groups are added up, and then a continuous sub-group corresponding to the minimum differences sum among the obtained differences sums is selected. For example, a sub-groups section corresponding to the minimum differences sum of continuous 5 difference values Dn is searched for.

$$Sum\_ 1 = D 1 + D 2 + D 3 + D 4 + D 5$$

$$Sum\_ 2 = D 2 + D 3 + D 4 + D 5 + D 6$$

$$Sum\_ 3 = D 3 + D 4 + D 5 + D 6 + D 7$$

$$…$$

$$Sum\_ 6 = D 6 + D 7 + D 8 + D 9 + D 10$$

$$…$$

For example, if Sum_{—}8 is the smallest among obtained Sum values, a section from D8 to D12, that is, a section from sub-group S8 to sub-group S12 is selected for calculating a slope.

FIG. 4 is a graph illustrating an example of a section that is selected as being appropriate to obtain a slope. Referring to FIG. 4, in a section from S8 to S12 obtained as described above, a straight line is obtained by the regression analysis and a slope can be obtained therefrom (Bold line of FIG. 4). The slope represents an enzymatic reaction rate. The slope can be calculated as described above.

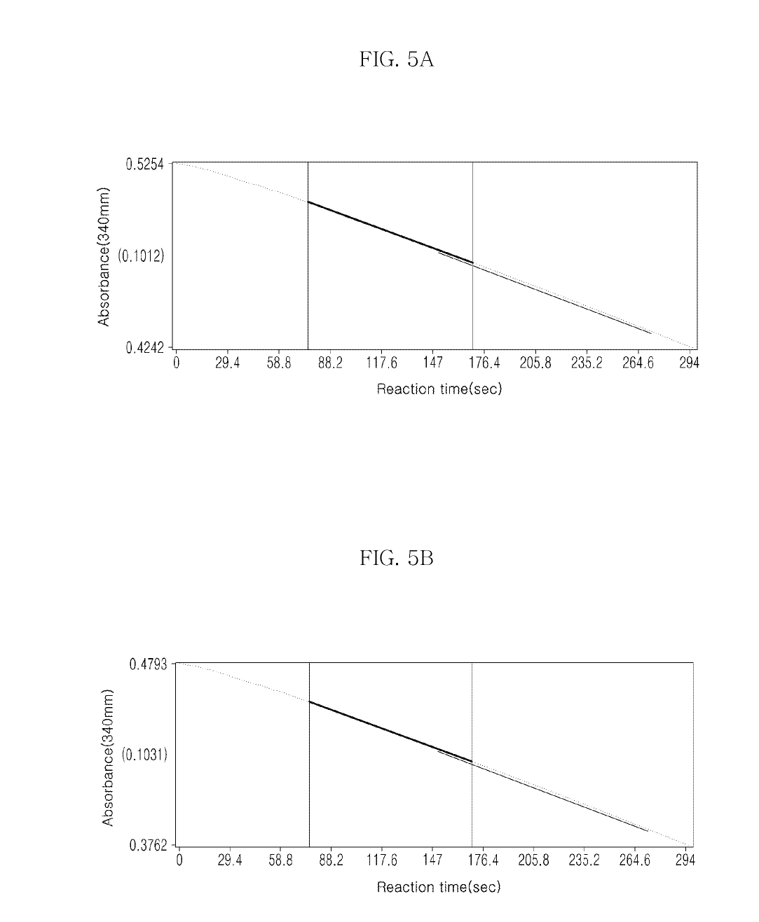

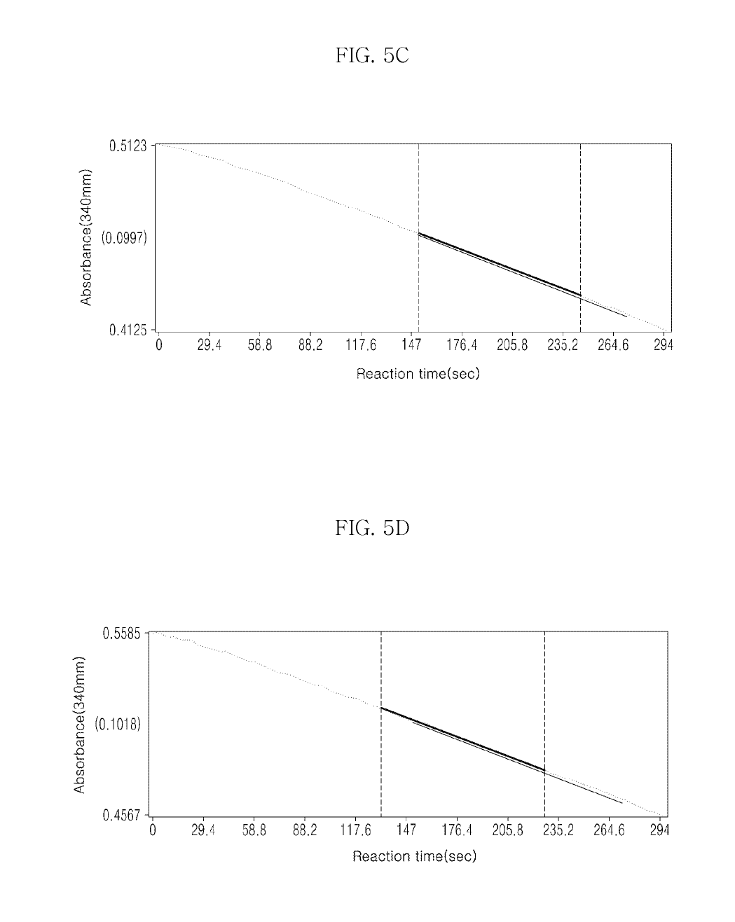

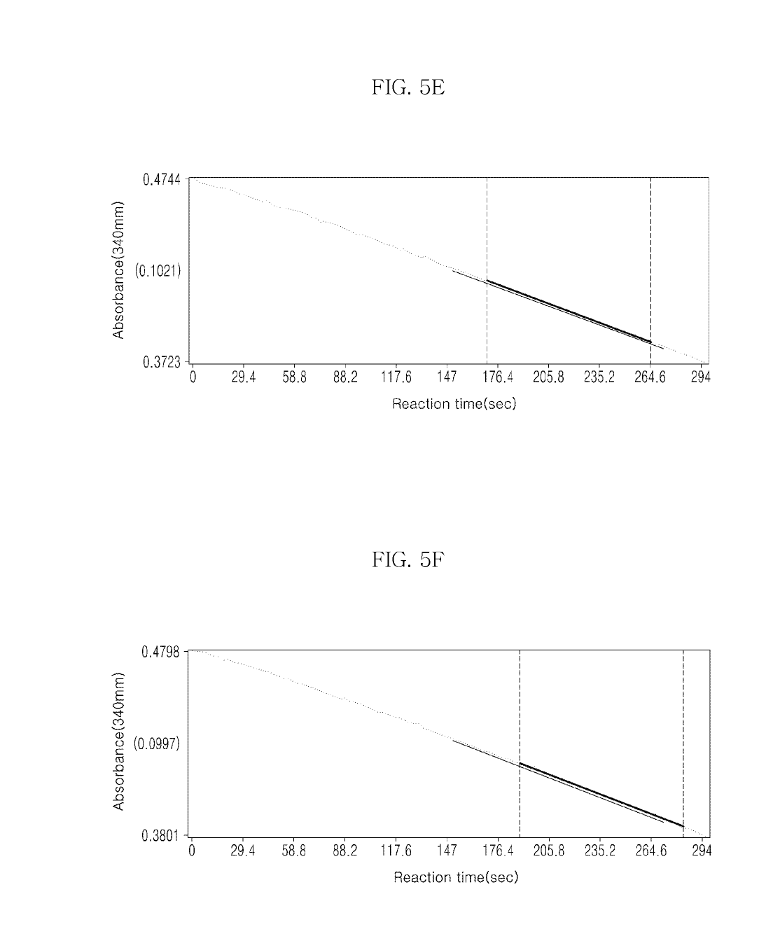

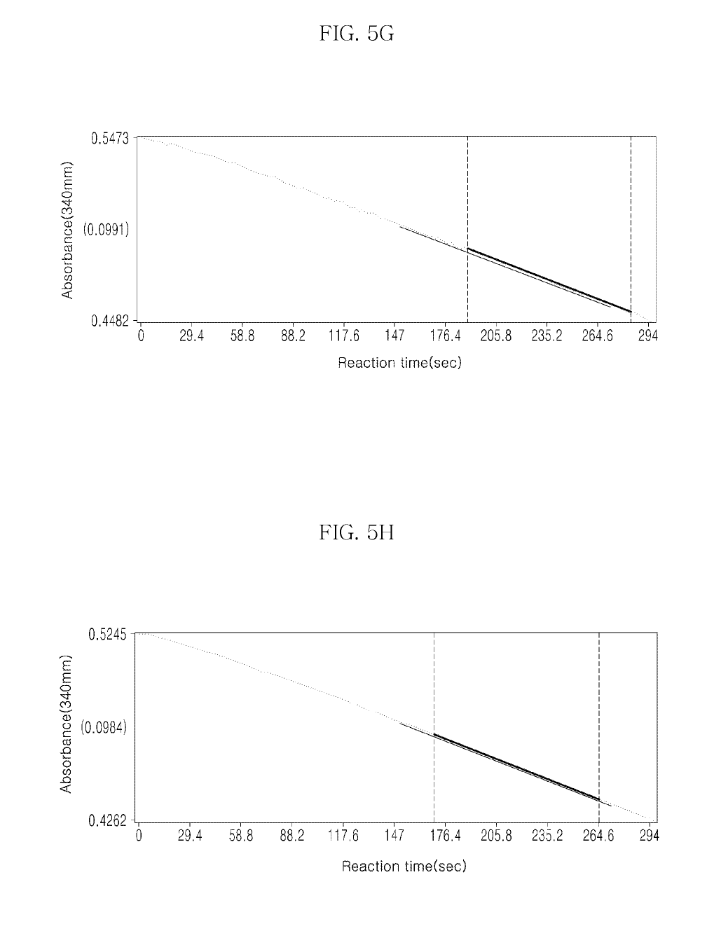

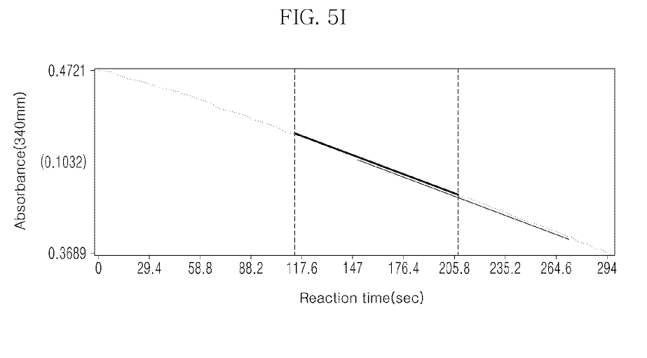

FIGS. 5A through 5I are graphs illustrating results obtained by assaying a sample (serum containing AST) 9 times using a method according to an exemplary embodiment. In this case, the optical signal profile was divided into 15 sub-groups, Sf was a middle value, and the number of continuous sub-groups was 5. % CV value of the obtained slope value was 0.98% (experimental group: bold line), and % CV value of a slope calculated using a conventional calculation method (a method of calculating a slope with respect to absorbance measured between 150 seconds and 270 seconds) was 1.53% (control group: thin line).

TABLE 1

| Number of | | |
|---|---|---|
| Experiments | Experimental Group | Control Group |
| 1 | −0.000352067 | −0.000362639 |
| 2 | −0.000356613 | −0.000368526 |
| 3 | −0.000355192 | −0.00036008 |
| 4 | −0.000361931 | −0.000364863 |
| 5 | −0.000360836 | −0.000357503 |
| 6 | −0.000361407 | −0.000356342 |
| 7 | −0.000361107 | −0.000354367 |
| 8 | −0.00035581 | −0.000354129 |
| 9 | −0.000360198 | −0.000367632 |
| % CV | 0.98 | 1.53 |

Accordingly, if the method as described above is used, a uniform enzymatic reaction rate can be obtained. In addition, if enzymatic reaction rates are calculated using the method as described above, the amount of an enzyme in a sample can be uniformly determined.

As described above, according to the method of calculating an enzymatic reaction rate according to the exemplary embodiment, an enzymatic reaction rate can be precisely, efficiently calculated.

By using a computer program product according to the exemplary embodiment as described above, an enzymatic reaction rate can be precisely, efficiently calculated.

According to a method of determining an amount of an enzyme in a sample according to the exemplary embodiment as described above, the amount of the enzyme in the sample can be precisely, efficiently measured.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

What is claimed is:

1. A method of calculating a reaction rate of a first substance, the method comprising:
obtaining an optical signal, with respect to a reaction time, of a mixture including the first substance and a second substance, wherein a reaction between the first substance and the second substance generates the optical signal;
dividing the optical signal into sub-groups of the optical signal based on a reaction time interval;
calculating a slope with respect to each sub-group of the optical signal and the reaction time;
obtaining a frequency of the calculated slope of each sub-group with respect to a slope value interval;
subtracting a slope value (Sn) of each sub-group from a slope value (Sf) that corresponds to a largest frequency value among the obtained frequency values, thereby obtaining differences between Sf and Sn;
adding up the differences with respect to a predetermined number of continuous sub-groups and selecting a continuous sub-group section that corresponds to a minimum differences sum among the added-up differences sums; and
calculating a slope of the selected continuous sub-group section and determining the calculated slope as a reaction rate of the first substance.

2. The method of claim 1, wherein the optical signal is a fluorescent signal.

3. The method of claim 1, wherein the slope is calculated by a regression analysis or a robust estimation.

4. The method of claim 1, further comprising displaying the determined reaction rate to a user.

5. The method of claim 1, wherein the a frequency of the calculated slope of each sub-group with respect to a slope value interval represents a number of sub-groups having a substantially same slope value.

6. The method of claim 1, wherein the slope value is a median slope value in each sub-group.

7. The method of claim 1, wherein the first substance is an enzyme and the second substance is a substrate of the enzyme.

8. A computer-readable recording medium having recorded thereon a program for executing the method of claim 1.

9. A method of determining an amount of a first substance in a sample, the method comprising:
calculating a reaction rate of the first substance with respect to a sample comprising the first substance by using the method of claim 1; and
comparing the calculated reaction rate with a reference reaction rate for a known concentration of the first substrate, in order to determine the amount of the enzyme.

10. The method of claim 9, wherein the sample is selected from the group consisting of blood, tissues, salvia, urine, and a body fluid.

11. The method of claim 9, wherein the first substance is an enzyme.

12. The method of claim 11, wherein the enzyme is alkaline phosphatase or aminotransferase.

13. A computer-readable recording medium having recorded thereon a program for executing the method of claim 9.
